(19) United States
(12) Patent Application Publication
(10) Pub. No.: US 2021/0057078 A1
(43) Pub. Date: Feb. 25, 2021

(54) SYSTEM, DEVICE, METHOD, AND PROGRAM FOR TREATING DISORDER TREATABLE BY BEHAVIOR MODIFICATION

(71) Applicant: CureApp, Inc., Tokyo (JP)

(72) Inventors: Shin Suzuki, Chuo-ku, Tokyo (JP); Masaki Aijima, Ota-ku, Tokyo (JP)

(73) Assignee: CureApp, Inc., Tokyo (JP)

(21) Appl. No.: 16/962,977

(22) PCT Filed: Jan. 18, 2018
(86) PCT No.: PCT/JP2018/001407

§ 371 Date: Jul. 17, 2020

(51) Int. Cl. G16H 20/70; G16H 10/60; G16H 50/70; G16H 70/20; G16H 50/20; G16H 50/50; G09B 19/00

(52) U.S. Cl. CPC G16H 20/70; G16H 10/60; G16H 50/70; G09B 19/00; G16H 50/20; G16H 50/50; G16H 70/20

(57) ABSTRACT

A system used for treating a disorder treatable by behavior change includes: a server; and a user terminal. Medical traits, associated with a disorder, for indicating medical traits of a patient are clustered into behavioral medical traits, knowledge-related medical traits, and cognitive medical traits. The server is configured to: store each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait; select a therapy to be executed; and transmit therapy information for the therapy thus selected.

### TECHNICAL FIELD

The present invention relates to a system, a device, a method, and a program for treating a disorder treatable by behavior change.

### BACKGROUND ART

In conventional healthcare, a physician can only treat a patient during a medical examination. Treatment provided during the medical examination includes acts such as surgery, procedures, and prescribing medicine, and many disorders are cured by such acts. On the other hand, there are also disorders treatable by changing daily behavior. For disorders and psychiatric disorders caused by lifestyle in particular, it is often the case that changing daily behavior is more effective rather than providing treatment through outpatient medical care. This is because lifestyle is not something found at a hospital which is not on a day-to-day basis, but something found at the “home” of the patient, which is on a day-to-day basis. Therefore, when it comes to treating a disorder caused by behavior in daily life, even a healthcare provider, such as a physician, cannot provide sufficient advice by merely providing explanation during medical examinations several times a month. For the patient as well, situations occur in which the patient does not know how to utilize, on a daily basis, the advice given during the medical examination.

### CITATION LIST

### Patent Literature

Patent Document 1: JP 2001-92876 A

### SUMMARY OF INVENTION

### Technical Problem

Patent Document 1 discloses a system configured to sequentially provide to an individual, on a daily basis, a behavior change message for improving a behavior detrimental to health on the basis of data collected from the individual. By using this system, the patient can receive a behavior change message once per day, and thus understand the behavior that should be adopted on that day. However, the system described in Patent Document 1 merely discloses providing a behavior change message solely on the basis of data collected from the individual, and does not provide a solution for providing effective therapy for behavior change.

### Solution to Problem

The present invention has been made in view of the problems described above, and has characteristics such as the following. That is, a system according to an embodiment of the present invention is a system used for treating a disorder treatable by behavior change. The system includes a server and a user terminal, wherein medical traits, associated with a disorder, for indicating medical traits of a patient are clustered into behavioral medical traits, knowledge-related medical traits, and cognitive medical traits, the server is configured to store each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait, select a therapy to be executed from among the one or more therapies associated with, from among the plurality of behavioral medical traits, a behavioral medical trait selected to be treated, and therapies associated with knowledge-related medical trait information and cognitive medical trait information associated with the behavioral medical trait thus selected, and transmit therapy information for the therapy thus selected, and the user terminal is configured to present information for the therapy on the basis of the therapy information received.

The server may be further configured to store a medical trait state indicating a state of each of the medical traits of each patient, a standard selection probability factor for each of the one or more therapies associated with each of the medical traits, and an individual selection probability factor for each of the medical traits of each patient, the individual selection probability factor for each of the medical traits may be determined on the basis of the medical trait state of each of the medical traits of the patient, and a selection probability of the therapy may be determined on the basis of the standard selection probability factor for the therapy and the individual selection probability factor for a medical trait of the medical traits associated with the therapy.

The individual selection probability factor may be further determined on the basis of a cluster factor, and the cluster factor may be determined per patient for each cluster of the medical traits.

The server may be further configured to store attributes of each patient, the attributes may include at least one of a gender, an age, or an occupation, and the cluster factor may be determined on the basis of the attributes.

The server may be further configured to acquire effectiveness information indicating whether a medical trait associated with the therapy thus selected has improved, update the medical trait state of the patient on the basis of the effectiveness information, and change the individual selection probability factor on the basis of the medical trait state thus updated.

The server may be further configured to acquire effectiveness information indicating whether a medical trait associated with the therapy thus selected has improved, and change a cluster factor of a cluster to which the medical trait belongs on the basis of the effectiveness information.

The server may be further configured to acquire effectiveness information indicating whether the medical trait associated with the therapy thus selected has improved, and change the standard selection probability factor for the therapy thus selected on the basis of the effectiveness information.

The server may be configured to, in the selection of the therapy, select two or more of the therapies, and transmit the therapy information for the two or more therapies thus selected. The user terminal may be configured to present information for the two or more therapies on the basis of the therapy information received, and transmit, to a server, user selection information indicating a therapy selected by a user from the two or more therapies of the information presented. The server may be configured to change at least the standard selection probability factor on the basis of the therapy selection information.

The standard selection probability factor thus changed may be the standard selection probability factor for each of the therapies associated with the medical traits belonging to the cluster of the medical traits associated with the therapy thus selected.

A server according to an embodiment of the present invention is a server used for treating a disorder treatable by behavior change where medical traits, associated with a disorder, for indicating medical traits of a patient are clustered into behavioral medical traits, knowledge-related medical traits, and cognitive medical traits, the server being configured to store each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait, select a therapy to be executed from among the one or more therapies associated with, from among the plurality of behavioral medical traits, a behavioral medical trait selected to be treated, and therapies associated with knowledge-related medical trait information and cognitive medical trait information associated with the behavioral medical trait thus selected, and transmit therapy information for the therapy thus selected.

A method according to an embodiment of the present invention is a method executed by a system used for treating a disorder treatable by behavior change where medical traits, associated with a disorder, for indicating medical traits of a patient are clustered into behavioral medical traits, knowledge-related medical traits, and cognitive medical traits, the system including a server and a user terminal, and the server being configured to store each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait, the method including the steps of selecting, by the server, a therapy to be executed from among the one or more therapies associated with, from among the plurality of behavioral medical traits, a behavioral medical trait selected to be treated, and therapies associated with knowledge-related medical trait information and cognitive medical trait information associated with the behavioral medical trait thus selected, and transmitting, by the server, therapy information for the therapy thus selected, and presenting, by the user terminal, information for the therapy on the basis of the therapy information received to execute the therapy.

A method according to an embodiment of the present invention is a method executed by a server used for treating a disorder treatable by behavior change where medical traits, associated with a disorder, for indicating medical traits of a patient are clustered into behavioral medical traits, knowledge-related medical traits, and cognitive medical traits, the system including a server and a user terminal, and the server being configured to store each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait, the method including the steps of selecting, by the server, a therapy to be executed from among the one or more therapies associated with, from among the plurality of behavioral medical traits, a behavioral medical trait selected to be treated, and therapies associated with knowledge-related medical trait information and cognitive medical trait information associated with the behavioral medical trait thus selected, and transmitting, by the server, therapy information for the therapy thus selected.

A program according to an embodiment of the present invention is a program configured by a set of programs used for treating a disorder treatable by behavior change where medical traits, associated with a disorder, for indicating medical traits of a patient are clustered into behavioral medical traits, knowledge-related medical traits, and cognitive medical traits, the set of programs being configured to cause one or more computers to perform storing each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait, selecting a therapy to be executed from among the one or more therapies associated with, from among the plurality of behavioral medical traits, a behavioral medical trait selected to be treated, and therapies associated with knowledge-related medical trait information and cognitive medical trait information associated with the behavioral medical trait thus selected, and presenting information for the therapy on the basis of therapy information for the therapy thus selected to execute the therapy.

A program according to an embodiment of the present invention is a program used for treating a disorder treatable by behavior change where medical traits, associated with a disorder, for indicating medical traits of a patient are clustered into behavioral medical traits, knowledge-related medical traits, and cognitive medical traits, the server being configured to store each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait, the program being configured to cause the server to perform selecting a therapy to be executed from among the one or more therapies associated with, from among the plurality of behavioral medical traits, a behavioral medical trait selected to be treated, and therapies associated with knowledge-related medical trait information and cognitive medical trait information associated with the behavioral medical trait thus selected, and transmitting therapy information for the therapy thus selected.

### Advantageous Effects of Invention

Through use of the present invention, it is possible to treat a disorder by effectively modifying a behavior of a patient. According to one embodiment, a behavior of a patient is effectively treated by clustering medical traits into behavioral medical traits, knowledge-related medical traits, and cognitive medical traits, defining relationships between each medical trait and relationships between medical traits and therapies, acquiring a state of the patient for each trait, and allowing the patient to select an appropriate therapy for a cause of an undesirable behavior. Furthermore, with use of the effectiveness information of the therapies, a more effective therapy can be provided by changing the selection probability of each therapy on the basis of the effectiveness information. With use of the user terminal of the patient, an appropriate therapy based on an ever-changing state of the patient can be provided. With use of the user terminal of a healthcare provider, it is possible to select a more appropriate therapy on the basis of the selection information of the healthcare provider.

### BRIEF DESCRIPTION OF DRAWINGS

FIG. 1 is a configuration diagram of a system according to an embodiment of the present invention.

FIG. 2 is a hardware configuration diagram of a user terminal according to an embodiment of the present invention.

FIG. 3 is a hardware configuration diagram of a server according to an embodiment of the present invention.

FIG. 4 is a functional block diagram of the user terminal according to an embodiment of the present invention.

FIG. 5 is a functional block diagram of the server according to an embodiment of the present invention.

FIG. 6 is a correlation diagram of medical traits and therapies according to an embodiment of the present invention.

FIG. 7 is a flowchart according to an embodiment of the present invention.

FIG. 8 is a flowchart according to an embodiment of the present invention.

### DESCRIPTION OF EMBODIMENTS

### First Embodiment

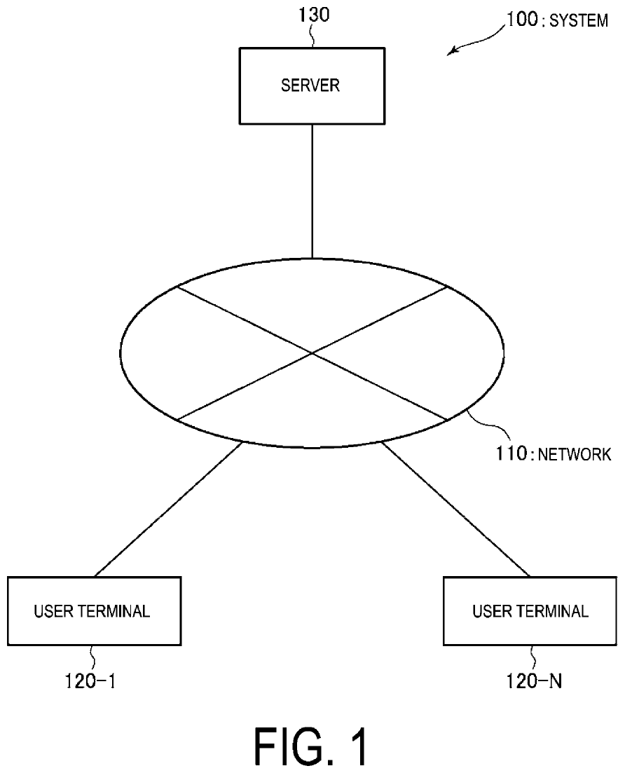

FIG. 1 illustrates an example of a system configuration diagram of the present invention. A system 100 is used for treating a disorder treatable by behavior change, and includes a network 110, and a user terminal 120 and a server 130 connected to the network 110.

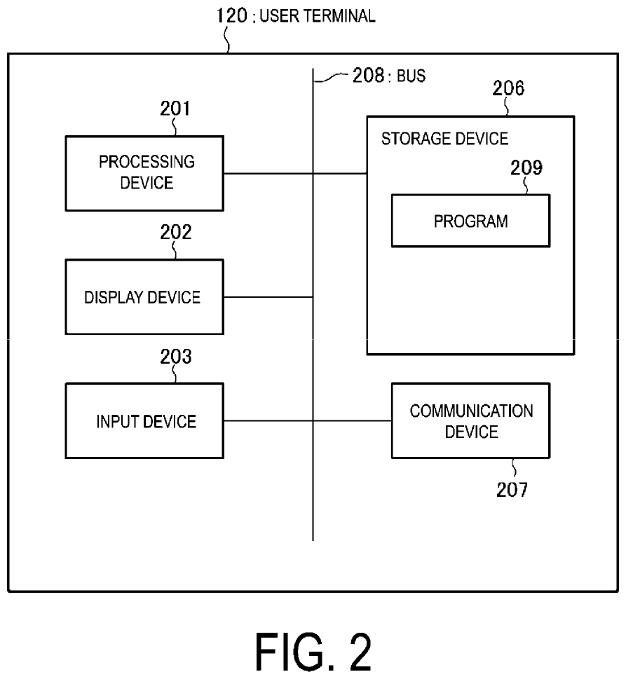

FIG. 2 illustrates an example of a hardware configuration diagram of the user terminal 120. The user terminal 120 is an electronic device including a processing device 201, a display device 202, an input device 203, a storage device 204, and a communication device 205. Each of these components is connected via a bus 208, but may be connected individually as needed. In the present embodiment, the user terminal 120 is a smartphone, but may be another electronic device, such as a mobile information terminal, a mobile phone, a tablet terminal, or a computer. A program 206 for implementing the present invention is stored in the storage device 204. The storage device 204 may be any storage device that is capable of storing information, such as a hard disk, a non-volatile memory, or a volatile memory. The communication device 205 preferably communicates with the server 130 via wireless communication such as Bluetooth (trade name) or a wireless local area network (LAN), but may be wired communication using an Ethernet (trade name) cable or the like.

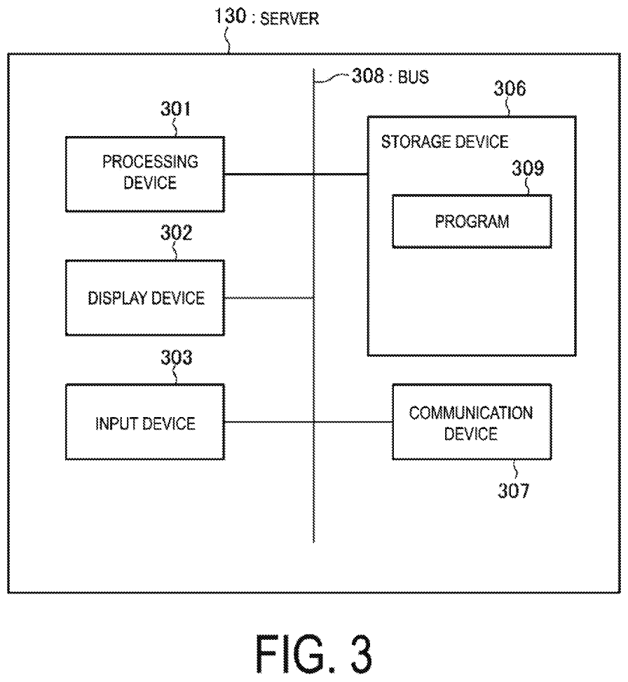

FIG. 3 illustrates an example of a hardware configuration diagram of the server 130. The server 130 includes a processing device 301, a display device 302, an input device 303, a storage device 304, and a communication device 305. Each of these components is connected via a bus 308, but may be connected individually as needed. The server 130 may be a computer, or may be a mobile information terminal, a mobile phone, a smartphone, or a tablet terminal. The display device 302 has a function of displaying information to a server user. The input device 303 has a function of receiving input from a user, such as a keyboard, a mouse, or the like. In a case where the server 130 is a smartphone or a tablet terminal, the display device 302 and the input device 303 may also be integrated into a touch panel. A program 306 for implementing the present invention is stored in the storage device 304. The storage device 304 may be any storage device that is capable of storing information, such as a hard disk, a non-volatile memory, or a volatile memory. The communication device 305 performs wired communication using an Ethernet (trade name) cable or the like, or wireless communication using mobile communication, Bluetooth (trade name), a wireless LAN, or the like, and connects to the user terminal 120.

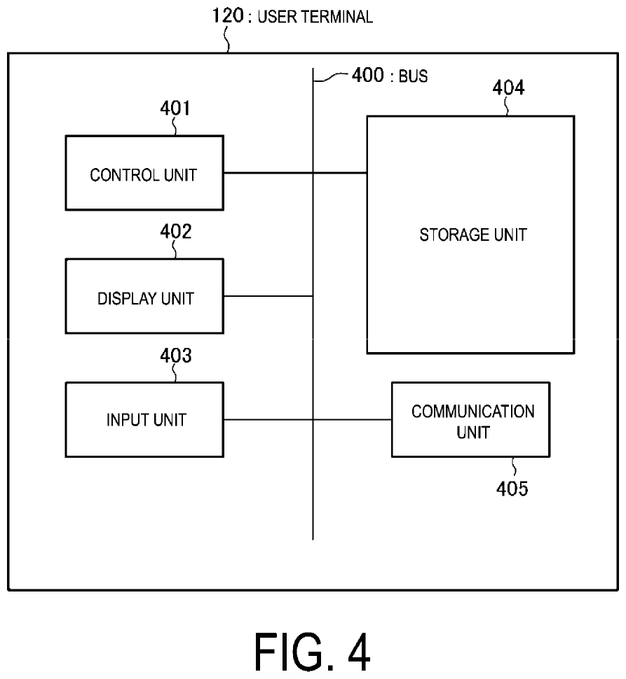

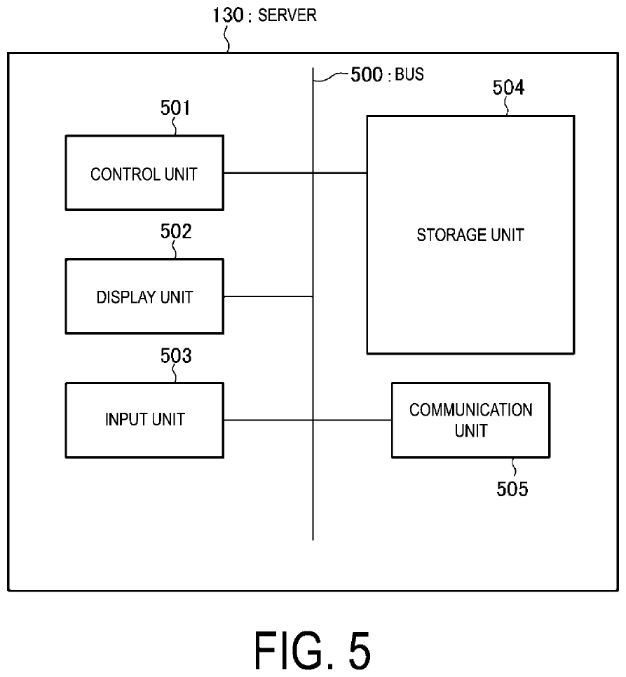

FIGS. 4 and 5 illustrate examples of functional block diagrams of the user terminal 120 and the server 130 of the present invention. The user terminal 120 includes a control unit 401, a display unit 402, an input unit 403, a storage unit 404, and a communication unit 405, and the server 130 includes a control unit 501, a display unit 502, an input unit 503, a storage unit 504, and a communication unit 505. The control units 401 and 501 each have a function of executing control, such as information processing. The display units 402 and 502 each have a function of displaying information so that the user can view the information. The input units 403 and 503 each have a function of receiving input from a user. The storage units 404 and 504 each have a function of storing tables, data, and the like. The communication units 405 and 505 each have a function of transmitting and receiving information to and from other devices. In the present embodiment, these functions are implemented by programs 209, 309 being executed in the processing devices 201 and 301 illustrated in FIGS. 2 and 3, and respective hardware and software operating in cooperation, but can be implemented by configuring an electronic circuit or the like for implementing each function.

In the present embodiment, the disorder treatable by behavior change is fatty liver, but may be any disorder treatable by behavior change, such as a so-called lifestyle disease such as hypertension, or a psychiatric disorder. The disorder need only be a physically undesirable state, and need not be a disorder in a medical sense. Treatment by behavior change includes preventive medicine. The term “patient” refers to a person who attempts to treat a disorder by behavior change using the present invention, and does not necessarily need to treat the disorder under the guidance of a healthcare provider.

Medical traits related to the disorder of the patient are categorized and clustered into behavioral medical traits (MTs), knowledge-related medical traits (MTs), and cognitive medical traits (MTs). The behavioral MTs are traits pertaining to the behavior of the patient associated with the disorder. The knowledge-related MTs are traits pertaining to the knowledge of the patient associated with the disorder to be treated, and the cognitive MTs are traits pertaining to the cognition of the patient associated with the disorder. Knowledge pertains to objective facts, whereas cognition is the way of thinking of the patient and is subjective. These states of each patient are referred to as behavioral medical trait states (MSs), knowledge-related medical trait states (MSs), and cognitive medical trait states (MSs).

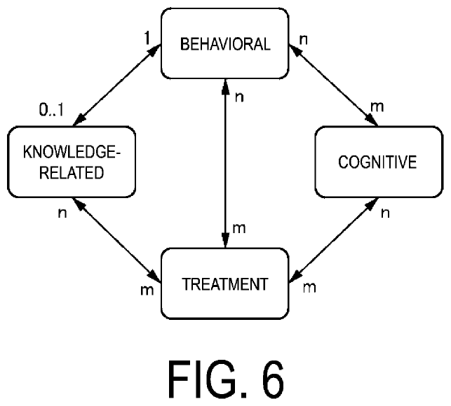

The behavioral MTs, the knowledge-related MTs, the cognitive MTs, and the therapies are given the correlation illustrated in FIG. 6. That is, one behavioral MT is associated with 0 to 1 knowledge-related MTs. In a case where the knowledge that can be associated with the behavioral MT is common knowledge that everyone knows, the knowledge can be omitted, and thus there may be a case where a knowledge-related MT is not associated with one behavioral MT. n behavioral MTs are associated with m cognitive MTs, and n behavioral MTs are associated with m therapies. n knowledge-related MTs are associated with m therapies, and n cognitive MTs are associated with m therapies. n and m are integers greater than or equal to 1. For convenience, the expressions n and m are uniformly used for each MT and therapy, but do not necessarily mean that each pair of n and m indicates the same pair of integers. Depending on the disorder, one behavioral MT can also be associated with two or more knowledge-related MTs. That is, n behavioral MTs may be associated with m knowledge-related MTs.

The present invention treats a disorder by behavior change, and therefore is intended to modify an undesirable behavior of the patient to a desirable one. The cause of the undesirable behavior of the patient is considered to be one or both of not having the correct knowledge associated with the behavior and not having the correct cognition associated with the behavior. Accordingly, an appropriate treatment for eliminating the cause of the undesirable behavior of the patient is carried out, thereby modifying the undesirable behavior of the patient to the desirable one. Further, therapies that directly modify a behavior itself to the desirable one, without modifying knowledge or cognition also exist. In the present invention, it is possible to define the correlation between each MT and therapy as illustrated in FIG. 6, and identify the therapy appropriate for each MT.

In order to start the information processing in the present embodiment, a Behavioral MT Table, a Knowledge-Related MT Table, a Cognitive MT Table, and a Therapy Table are generated. Furthermore, in order to define the correlation between each MT and therapy illustrated in FIG. 6, a Behavioral MT-Knowledge-Related MT Relationship Table, a Behavioral MT-Cognitive MT Relationship Table, a Behavioral MT-Therapy Relationship Table, an Intention MT-Therapy Relationship Table, and a Cognitive MT-Therapy Relationship Table are generated. Each table of a working example is illustrated below.

TABLE 1
Behavioral MT Table

| Behavioral MT ID | Description | Possible State |
|---|---|---|
| eatAtOnce | May binge on opened | 5: Strongly agree |
| | sweets until all are gone | 4: Somewhat agree |
| | | 3: Not sure |
| | | 2: Somewhat disagree |
| | | 1: Strongly disagree |
| cookTooMuch | Cooks too much food | 5: Strongly agree |
| | | 4: Somewhat agree |
| | | 3: Not sure |
| | | 2: Somewhat disagree |
| | | 1: Strongly disagree |

TABLE 2
Knowledge-Related MT Table

| Knowledge- | | |
|---|---|---|
| Related MT ID | Description | Possible State |
| EatAtOnceIntent | To binge on sweets | 5: Strongly agree |
| | is not a bad thing | 4: Somewhat agree |
| | | 3: Not sure |
| | | 2: Somewhat disagree |
| | | 1: Strongly disagree |
| cookTooMuch | To cook too much | 5: Strongly agree |
| | is not a bad thing | 4: Somewhat agree |
| | | 3: Not sure |
| | | 2: Somewhat disagree |
| | | 1: Strongly disagree |

TABLE 3
Cognitive MT Table

| Cognitive MT ID | Description | Possible State |
|---|---|---|
| noLeave | To leave or throw away | 5: Strongly agree |
| | food is a bad thing | 4: Somewhat agree |
| | | 3: Not sure |
| | | 2: Somewhat disagree |
| | | 1: Strongly disagree |
| adequateAmount | Shops and products are | 5: Strongly agree |
| | designed to be favorable | 4: Somewhat agree |
| | in both taste and quantity | 3: Not sure |
| | | 2: Somewhat disagree |
| | | 1: Strongly disagree |
| worryAboutShortness | Worrying about not | 5: Strongly agree |
| | having enough | 4: Somewhat agree |
| | | 3: Not sure |
| | | 2: Somewhat disagree |
| | | 1: Strongly disagree |
| mottainaiIngredient | Wanting to use up | 5: Strongly agree |
| | the food in the house | 4: Somewhat agree |
| | and not waste it | 3: Not sure |
| | | 2: Somewhat disagree |
| | | 1: Strongly disagree |
| noRestriction | Not wanting to worry | 5: Strongly agree |
| | about food choices | 4: Somewhat agree |
| | or thinking too much | 3: Not sure |
| | | 2: Somewhat disagree |
| | | 1: Strongly disagree |

TABLE 4
Therapy Table

| Therapy ID | Description |
|---|---|
| trush | Just throw them away! |
| calorieEstimate | Estimate the approximate calories in a correct manner! |
| decisionFatigue | People are not very rational. If we do not make decisions |
| | in advance, we tend to eat more than we need. |
| BeyondGoodandEvil | It is more important to treat the disorder that you yourself |
| | are facing than assume blind values of good and evil. |
| specialization | The world is uniformly designed, but we are each an |
| | individual. Each person should think about and choose |
| | his or her own way of life. |
| justRightIngredient | Buy the right amount of food. |
| opportunityCost | There are limited opportunities to eat this day. It would |
| | be a shame if you do not cherish eating. |
| postSatisfactionOverEating | The feeling of satisfaction after eating is met when you |
| | have eaten until you are no longer hungry. You will |
| | not feel well if you eat too much. |
| calorieAccounting | It is not worth using your right to eat whatever you |
| | want in one day, eating what you do not need. |
| healthAccounting | It is not worth eating something cheap and bad for you |
| | today to only suffer healthcare expenses and an |
| | uncomfortable life in the future. |

TABLE 5
Behavioral MT-Knowledge-Related
MT Relationship Table

| Behavioral MT ID | Knowledge-Related MT ID |
|---|---|
| eatAtOnce | EatAtOnceIntent |
| cookTooMuch | cookTooMuchIntent |

TABLE 6
Behavioral-Cognitive MT Relationship Table

| Behavioral MT ID | Cognitive MT ID |
|---|---|
| eatAtOnce | noLeave |
| eatAtOnce | adequateAmount |
| cookTooMuch | noRestriction |
| cookTooMuch | worryAboutShortness |
| cookTooMuch | mottainaiIngredient |

TABLE 7
Behavioral MT-Therapy Relationship Table

| | | Standard |
|---|---|---|
| | | Selection |
| | | Probability |
| Behavioral MT ID | Therapy ID | Factor |
| eatAtOnce | trush | 1.2 |
| cookTooMuch | justRightIngredient | 1.2 |

TABLE 8
Knowledge-Related MT-Therapy Relationship Table

| | | Standard |
|---|---|---|
| | | Selection |
| | | Probability |
| Knowledge-Related MT ID | Therapy ID | Factor |
| EatAtOnceIntent | calorieEstimate | 1.0 |
| EatAtOnceIntent | decisionFatigue | 0.8 |
| cookTooMuchIntent | calorieEstimate | 0.9 |

TABLE 9
Cognitive MT-Therapy Relationship Table

| | | Standard |
|---|---|---|
| | | Selection |
| | | Probability |
| Cognitive MT ID | Treatment ID | Factor |
| noLeave | BeyondGoodandEvil | 0.9 |
| adequateAmount | specialization | 0.9 |
| noRestriction | opportunityCost | 0.7 |
| noRestriction | postSatisfactionOverEating | 0.6 |
| worryAboutShortness | postSatisfactionOverEating | 0.7 |
| mottainaiIngredient | calorieAccounting | 1.2 |
| mottainaiIngredient | healthAccounting | 0.8 |

The Behavioral MT Table, the Knowledge-Related MT Table, and the Cognitive MT Table include MT IDs, descriptions, and possible states. The MT IDs are each an identifier for referencing an MT, and the descriptions are each a detailed description of the MT identified by the MT ID. The possible states each indicate a possible state of the MT. For example, the MT having the behavioral MT ID “eatAtOnce” is a trait pertaining to the behavior “May binge on opened sweets until all are gone” of the patient, and is indicated as having five possible states on a scale of “1: Strongly disagree” to “5: Strongly agree”. When the patient binges on opened sweets until all are gone, the caloric intake is likely to be excessive, and thus the behavior is not desirable for the treatment of fatty liver. Accordingly, in a case where the patient is in the state “5: Strongly agree”, this indicates that modification is required.

The knowledge-related MT is a trait pertaining to knowledge in association with the disorder. For example, the MT having the knowledge-related MT ID “EatAtOnceIntent” indicates the trait pertaining to the knowledge “To binge on opened sweets is not a bad thing.” That is, the MT indicates a trait of the patient pertaining to whether he or she has the correct knowledge that bingeing on sweets is not a desirable behavior for the treatment of fatty liver, and the possible states indicate how accurately the patient has that knowledge. The state “5: Strongly agree” indicates that the patient lacks the knowledge that bingeing on sweets is a bad thing, and is a state in which modification is required.

The cognitive MT is a trait pertaining to the cognition of the patient in association with the disorder. For example, the MT of the cognitive MT ID “noLeave” indicates a trait pertaining to the cognition “To leave or throw away food is a bad thing” and the possible states indicate how strongly the patient has the cognition “To leave or throw away food is a bad thing”. The cognition that not cleaning your plate is a bad thing is likely to lead to behavior resulting in excessive caloric intake and is not a desirable cognition for the treatment of fatty liver. The state “5: Strongly agree” indicates that the patient has a strong cognition that to leave or throw away food is a bad thing, and is a state in which modification is required.

The Therapy Table includes therapy IDs and descriptions. The therapy IDs are each an identifier for referencing a therapy, and the descriptions are each a detailed description of the therapy. The therapy IDs should be associated with the MT IDs, and information for modifying the associated medical trait to a desirable state is included as the description. Here, the description is information that serves as the basis of the message presented to the patient, and is, for example, “Just throw them away!” for the therapy ID “trush”.

Next, the Behavioral MT-Knowledge-Related MT Relationship Table, the Behavioral-Cognitive MT Relationship Table, the Behavioral MT-Therapy Relationship Table, the Knowledge-Related MT-Therapy Relationship Table, and the Cognitive MT-Therapy Relationship Table are tables indicating the correlation between the MTs and the correlation between the MTs and the therapies illustrated in FIG. 6. The tables each include MT IDs or therapy IDs and are associated with each other.

For example, the behavioral MT ID “eatAtOnce” in the Behavioral MT-Knowledge-Related MT Relationship Table is associated with the knowledge-related MT ID “EatAtOnceIntent”. This indicates that the behavioral MT ID “eatAtOnce” indicates a trait pertaining to the behavior of whether the patient “May binge on opened sweets until all are gone”, and this behavioral MT is associated with the presence or absence of the knowledge “To binge on sweets is not a bad thing” identified by the knowledge-related MT ID “EatAtOnceIntent”. A patient in the state “5: Strongly agree” for the trait of whether he or she “May binge on opened sweets until all are gone” is thought to binge on sweets until all are gone due to the mistaken knowledge that to binge on sweets is not a bad thing. On the other hand, a patient in the state “1: Strongly disagree” is thought to binge on sweets for another reason. These behavioral MTs and knowledge-related MTs are associated with each other to illustrate such relationships.

Each MT-Therapy Relationship Table associates MT IDs with therapy IDs to identify the therapies for each MT. For example, the therapy ID “trush” is associated with the behavioral MT ID “eatAtOnce” in the Behavioral MT-Therapy Relationship Table, indicating that the behavioral therapy “Just throw them away!” is applicable as a therapy for improving the trait “May binge on opened sweets until all are gone” of the behavioral MT ID “eatAtOnce”. The therapy ID “BeyondGoodandEvil” is associated with the cognitive MT ID “noLeave” in the Cognitive MT-Therapy Relationship Table, indicating that the cognitive therapy “It is more important to treat the disorder that you yourself are facing than assume blind values of good and evil”, which indicates the correct way of thinking, is applicable as a therapy for improving the trait “To leave or throw away food is a bad thing” of the cognitive MT ID “noLeave”.

Furthermore, each MT-Therapy Relationship Table includes a standard selection probability factor. This is a factor for determining the probability of selection of a therapy ID associated with the MT ID, and is a standard applied to all patients. The standard selection probability factor can be set in advance by a healthcare provider, a system provider, or the like, and can be subsequently updated on the basis of the actual effectiveness in all patients, or the like. Highly effective therapies are set to be more likely selected.

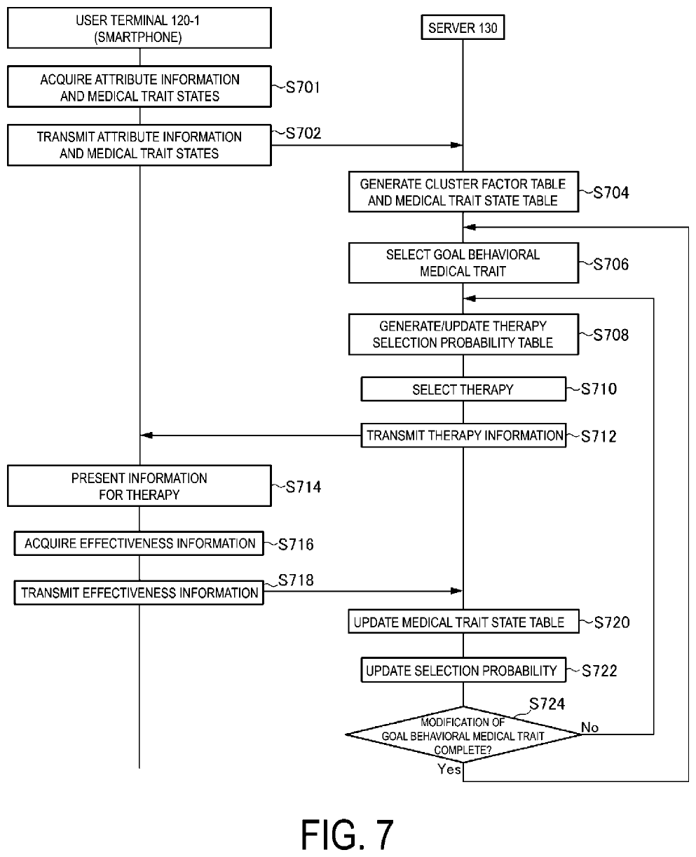

In the present embodiment, each of the tables described above is stored in the storage unit 504 of the server 130. These tables are then used to execute the processing of selecting the appropriate therapy for each patient. The operation of the user terminal 120-1 and the server 130 according to the present embodiment is described below using FIG. 7. Here, the user is a patient, and a smartphone of the patient is used as the user terminal 120-1. An application for implementing the present invention is pre-installed in the smartphone 120.

First, the control unit 401 of the smartphone 120 acquires attribute information and the medical trait states (MSs) of the patient on the basis of input by the patient via the input unit 403 (S701). The attribute information indicates patient information such as a gender, an age, and an occupation. The medical trait states indicate the individual states of the patient for each MT. The medical trait states can be acquired through interaction with a bot incorporated into the application, for example. According to one preferred aspect, at the timing when the application is installed and treatment is initiated, the control unit 401 displays predetermined questions on the display unit 402 and receives patient responses to the questions from the input unit 403, thereby acquiring the medical trait states of the patient at that point in time. For example, the application presents to patient A the question, “Mr. A, do you agree that ‘To leave or throw away food is a bad thing’?” along with the response options “5: Strongly agree, 4: Somewhat agree, 3: Not sure, 2: Somewhat disagree, 1: Strongly disagree”, prompting a response from patient A. In a case where patient A strongly agrees, patient A enters “5” via the input unit 403 in response. Further, because each MT of the patient is modified by practice of the present invention, preferably the patient interacts with the bot again to update the medical trait states of the patient.

The control unit 401 of the user terminal 120-1 transmits the attribute information and the medical trait states of the patient input via the communication unit 405 to the server 130 via the network 110 (S702). The attribute information and the medical trait states may be input via the user terminal 120 of the healthcare provider and transmitted to the server 130. A portion of the medical trait states may be input by the user terminal 120 of the healthcare provider and transmitted to the server 130 while the other portion is input from the user terminal 120 of the patient and transmitted to the server 130.

The server 130 generates a Cluster Factor Table and a Medical Trait State Table for the patient on the basis of the received medical trait states of the patient (S704). In a case where not all states pertaining to the medical traits have been acquired, the medical trait state “3: Not sure” can be input by default, for example, for those medical traits of which states are not acquired. In the present embodiment, the Medical Trait State Table of the patient includes an individual selection probability factor. The individual selection probability factor is one of the factors used in calculating the selection probability of each therapy, and is calculated here by Equation (1) below.

INDIVIDUAL SELECTION PROBABILITY FACTOR=MEDICAL TRAIT STATE×CLUSTER FACTOR Equation 1

Here, cluster factors are set for each patient. A cluster factor is determined on the basis of which cluster, behavioral MTs, knowledge-related MTs, or cognitive MTs, results in effective treatment for the patient. Depending on the patient, therapies for behavioral MTs may exhibit more effectiveness while therapies for cognitive MTs may not be very effective. In such a case, the cluster factor is set so that therapies for behavioral MTs are more likely selected. This cluster factor may be set in advance by the healthcare provider or the like, or may be automatically set on the basis of the attributes. For example, in a case where, for males, therapies for behavioral MTs exhibit more effectiveness, then the cluster factor is set high for a patient having an attribute of male. Examples of the Cluster Factor Table and the Medical Trait State Table for patient A are illustrated in the tables below.

TABLE 10
Cluster Factor Table of Patient A

| | Cluster |
|---|---|
| Cluster | Factor |
| Behavioral MTs | 1.2 |
| Knowledge-related MTs | 1.0 |
| Cognitive MTs | 0.8 |

TABLE 11
Medical Trait State Table of Patient A

| | | | | Individual |
|---|---|---|---|---|
| | | | | Selection |
| | | | Cluster | Probability |
| MT ID | Cluster Type | State | Factor | Factor |
| eatAtOnce | Behavioral MTs | 5 | 1.2 | 6.0 |
| EatAtOnceIntent | Knowledge-related | 5 | 1.0 | 5.0 |
| | MTs | | | |
| noLeave | Cognitive MTs | 3 | 0.8 | 2.4 |
| cookTooMuch | Behavioral MTs | 4 | 1.2 | 4.8 |
| cookTooMuchIntent | Knowledge-related | 1 | 1.0 | 1.0 |
| | MTs | | | |
| noRestriction | Cognitive MTs | 3 | 0.8 | 2.4 |
| worryAboutShortness | Cognitive MTs | 1 | 0.8 | 0.8 |
| mottainaiIngredient | Cognitive MTs | 4 | 0.8 | 3.2 |

The cluster factors of each cluster of behavioral MTs, knowledge-related MTs, and cognitive MTs of patient A are set to 1.2, 1.0 and 0.8 on the basis of attributes, as illustrated in the Cluster Factor Table (Table 10). The Medical Trait State Table includes MT IDs, cluster types, states, cluster factors, and individual selection probability factors. The MT IDs are each an ID of a medical trait, and the cluster types each indicate the cluster type to which the MT ID belongs. The states are each a state of the medical trait and are determined on the basis of the medical trait states transmitted from the user terminal 120. The cluster factors are each extracted from the Cluster Factor Table on the basis of the cluster type of the MT ID, and the individual selection probability factors are each calculated from the medical trait state and the cluster factor on the basis of Equation (1).

For example, for the MT ID “eatAtOnce”, the table indicates that the cluster type is “behavioral MT” and the medical trait state is “5”, that is, “5: Strongly agree”, on the basis of input by the patient. Then, the table indicates that the cluster factor is input as “1.2” on the basis of the Cluster Factor Table of Patient A, and the individual selection probability factor “6.0” is calculated by multiplying the cluster factor 1.2 by the medical trait state 5.

Next, in the server 130, from among the behavioral medical traits (MTs), a goal behavioral medical trait to be treated is selected (S706). For example, the behavioral MT ID “eatAtOnce” is a trait pertaining to whether the patient “May binge on opened sweets until all are gone.” Selection as the goal behavioral MT means that this behavioral MT is selected as the MT to be treated with the goal of achieving the desirable state of not bingeing on opened sweets until all are gone.

The goal behavioral MT can be selected by various methods. In one suitable working example, the patient selects the goal behavioral MT from behavioral MTs that are easily achievable. This is because gaining a successful experience can increase motivation to improve lifestyle habits. For example, the behavioral MT having the least number of cognitive MTs to be modified can be easily achieved. In a case where one of the two behavioral MTs of eatAtOnce and cookTooMuch is to be selected, the number of cognitive MTs associated with each MT is determined with reference to the Behavioral MT-Cognitive MT Relationship Table. Here, the number of cognitive MTs associated with eatAtOnce is two and the number of cognitive MTs associated with cookTooMuch is three, and therefore eatAtOnce, which is associated with less cognitive MTs, can be selected first as the goal behavioral MT. Alternatively, while the state of the behavioral MT of the patient is in a more desirable state, the MT for which the value of the state (1 to 5) is lowest can be selected, or the MT for which the average value of the medical trait states of the knowledge-related MT and the cognitive MT is lowest can be selected, for example.

Next, the knowledge-related MTs, the cognitive MTs, the therapies, and the medical trait states of patient A associated with the selected goal behavioral MT are acquired from each table to generate a Therapy Selection Probability Table for patient A (S708). To generate the Therapy Selection Probability Table, the selection probabilities of the therapies are determined. The selection probabilities of the therapies are each determined on the basis of the standard selection probability factor and the individual selection probability factor specific to the patient, for each therapy. An example of the Therapy Selection Probability Table for patient A for eatAtOnce with eatAtOnce selected as the goal behavioral MT in the present embodiment is illustrated below.

TABLE 12
Therapy Selection Probability Table of Patient A (Behavioral MT = eatAtOnce)

| | | Standard | Individual | Comprehensive | |
|---|---|---|---|---|---|
| Knowledge-related | | Selection | Selection | Selection | |
| MT/Cognitive | | Probability | Probability | Probability | Selection |
| MT ID | Therapy ID | Factor | Factor | Factor | Probability |
| — | trush | 1.2 | 6.0 | 7.20 | 0.335 |
| EatAtOnceIntent | calorieEstimate | 1.0 | 5.0 | 5.00 | 0.233 |
| EatAtOnceIntent | decisionFatigue | 0.8 | 5.0 | 4.00 | 0.186 |
| noLeave | BeyondGoodandEvil | 0.9 | 2.4 | 2.16 | 0.101 |
| noRestriction | opportunityCost | 0.7 | 2.4 | 1.68 | 0.078 |
| noRestriction | postSatisfactionOverEating | 0.6 | 2.4 | 1.44 | 0.067 |

The Therapy Selection Probability Table includes knowledge-related MT/cognitive MT IDs, therapy IDs, standard selection probability factors, individual selection probability factors, comprehensive selection probability factors, and selection probabilities. The knowledge-related MT/cognitive MT IDs each indicate the knowledge-related MT/cognitive MT ID associated with the goal behavioral MT “eatAtOnce” for extracting the therapy ID. A hyphen (“-”) entered for the knowledge-related MT/cognitive MT ID means that the therapy ID is a therapy ID directly associated with the behavioral MT “eatAtOnce”. The therapy IDs are each a therapy ID directly associated with the goal behavioral MT, or a therapy ID associated with a knowledge-related MT or cognitive MT ID associated with the goal behavioral MT. The comprehensive selection probability factors are each determined on the basis of the standard selection probability factor and the individual selection probability factor, and the selection probability is determined on the basis of the determined comprehensive selection probability factor. The therapy ID for execution is selected from the therapy IDs included in the Therapy Selection Probability Table on the basis of the selection probabilities (S710).

While there are a variety of techniques for the method for generating the Therapy Selection Probability Table, herein first the therapy ID “trush” directly associated with the goal behavioral MT “eatAtOnce” and the standard selection probability factor thereof (1.2) are acquired with reference to the Behavioral MT-Therapy Table (Table 7), and the individual selection probability factor (6.0) of the behavioral MT “eatAtOnce” is acquired with reference to the Medical Trait State Table of Patient A (Table 11). Furthermore, the knowledge-related MT “EatAtOnceIntent” associated with the goal behavioral MT “eatAtOnce” is acquired from the Behavioral MT-Knowledge-Related MT Association Table (Table 5), the therapies “calorieEstimate” and “decisionFatigue” associated with the acquired knowledge-related MT and the standard selection probability factors thereof (1.0 and 0.8) are acquired from the Knowledge-Related MT-Therapy Relationship Table (Table 8), and the individual selection probability factors (5.0) are acquired with reference to the MT ID “EatAtOnceIntent” in the Medical Trait State Table of Patient A (Table 11). Similarly, the therapies for cognitive MTs associated with the goal behavioral MT and the standard selection probability factors thereof are acquired from the Behavioral MT-Cognitive MT Association Table (Table 6) and the Cognitive MT-Therapy Relationship Table (Table 9), and the individual selection probability factors are acquired with reference to the MT IDs in the Medical Trait State Table of Patient A (Table 11).

A comprehensive selection probability factor Fn and a selection probability Pn are calculated by the equations below.

$$\begin{matrix} Equation 2 & \\ COMPREHENSIVE SELECTION PROBABILITY FACTOR {F}_{n} = STANDARD SELECTION PROBABILITY FACTOR {FS}_{n} = INDIVIDUAL SELECTION PROBABILITY FACTOR {FD}_{o} & ( 2 ) \\ Equation 3 & \\ SELECTION PROBABILITY {P}_{n} = \frac{COMPREHENSIVE PROBABILITY FACTOR {F}_{n}}{\begin{matrix}{\sum }_{k = 1}^{N} COMPREHENSIVE \\ SELECTION PROBABILITY FACTOR {F}_{k}\end{matrix}} & ( 3 )\end{matrix}$$

Here, given therapy numbers are assigned to therapies starting from the top of the Therapy Selection Probability Table (Table 12), n is the therapy number of the therapy for which the selection probability is to be calculated. The denominator of the right side of Equation (3) is the sum of the comprehensive selection probability factors of all therapies, and N is the number of selectable therapies (6 in the present embodiment).

For example, in the Therapy Selection Probability Table of Patient A (Behavioral MT ID=eatAtOnce) (Table 12), the comprehensive selection probability of the therapy ID “trush” is 7.20, which is calculated by multiplying the individual selection probability factor 6.0 by the standard selection probability factor 1.2. Then, the selection probability 0.335 is calculated by dividing the comprehensive selection probability 7.20 of the therapy ID “trush” by the sum of the comprehensive selection probabilities for all therapies in the Therapy Selection Probability Table.

Next, the control unit 501 of the server 130 selects a therapy for execution on the basis of the selection probabilities in the Therapy Selection Probability Table (S710), and transmits the therapy information for the selected therapy to the user terminal 120 via the communication unit 505 (S712). Here, the selection of the therapy is made by selecting a therapy ID on the basis of the selection probabilities of the Therapy Selection Probability Table of the patient. The therapy information indicates the information presented to the user for the selected therapy, and here includes the description for the selected therapy ID.

For example, in a case where “trush” is selected as the therapy ID for the behavioral MT ID “eatAtOnce,” the control unit 501 of the server 130 acquires the description “May binge on opened sweets until all are gone” for the behavioral MT ID “eatAtOnce” with reference to the Behavioral MT Table (Table 1) stored in the storage unit 504, and further acquires the information of the description “Just throw them away!” of the therapy “trush” with reference to the Therapy Table (Table 4). Then, the therapy information is generated on the basis of this information. For example, the message “To ensure that you do not binge on opened sweets until all are gone, just throw them away!” is generated and included in the therapy information.

When the control unit 401 of the user terminal 120-1 receives the therapy information, the information for the therapy is presented on the display unit 402 on the basis of the therapy information (S714). Here, the message “To ensure that you do not binge on opened sweets until all are gone, just throw them away!” is displayed on the display unit 402. The therapy information can also be presented to the patient by audio by using an output unit such as a speaker, or by another method.

A patient presented with the therapy executes the presented therapy to modify the behavior. For example, the behavior of patient A for which the state pertaining to the behavioral MT “Binges on opened sweets until all are gone” is “5: Strongly agree” and to whom the message “To ensure that you do not binge on opened sweets until all are gone, just throw them away!” is presented is expected to be modified to discarding remaining sweets without bingeing even if the sweets are opened. This makes it possible to suppress caloric intake.

Subsequently, the control unit 401 of the user terminal 120-1 executes an effectiveness information acquisition step (S716), and transmits the acquired effectiveness information to the server 130 (S718). For example, after a predetermined period has elapsed following presentation of the information for the therapy, the query message “Do you still binge on opened sweets until all are gone?” to confirm effectiveness, along with response options “5: Strongly agree, 4: Somewhat agree, 3: Not sure, 2: Somewhat disagree, 1: Strongly disagree” are presented on the display unit 402, and the effectiveness information is acquired by reception of a selection input from the patient.

In a case where the presented therapy is associated with a knowledge-related MT and a cognitive MT, the effectiveness can be confirmed by confirmation that the knowledge-related MT and the cognitive MT have been modified. Because it is expected that the goal behavioral MT is modified by modification of the knowledge-related MT and the cognitive MT, preferably the effectiveness information of the goal behavioral MT is acquired. As illustrated in FIG. 6, one therapy can be associated with a plurality of MTs. Accordingly, the medical trait states for other MTs associated with the therapy applied to the patient may also be acquired and updated.

The control unit 501 of the server 130, upon acquisition of the effectiveness information, updates the Medical Trait State Table on the basis of the effectiveness information (S720). For example, for the effectiveness of the therapy ID “trush” as a treatment for the behavioral MT ID “eatAtOnce”, the state of the MT ID “eatAtOnce” in the Behavioral Medical Trait State Table of Patient A is changed to “1” in a case where the response to the question “Do you still binge on opened sweets until all are gone?” is “1: Strongly disagree”.

Furthermore, the standard selection probability factor for the therapy is increased in a case where the medical trait state is improved, and decreased in a case where there is no effect (S722). With the standard selection probability factor modified on the basis of the actual treatment results of all users, the probability that a more effective therapy will be selected is increased, enabling more effective treatment.

The standard selection probability factor may be changed by changing the standard selection probability factors for all therapies, or by changing only the relationship with the MT confirmed as effective. For example, the therapy ID “postSatisfactionOverEating” is associated with both cognitive MT IDs “noRestriction” and “worryAboutShortness” in Table 9. In a case where the therapy ID “postSatisfactionOverEating” is selected in relationship to the cognitive MT ID “noRestriction” of the two cognitive MT IDs, and is confirmed as effective, only the standard selection probability factor “0.6” of the therapy ID “postSatisfactionOverEating” associated with the cognitive MT ID “noRestriction” may be increased, or the standard selection probability factor “0.7” associated with the cognitive MT ID “worryAboutShortness” may also be increased.

The standard selection probability factor can also be changed on a per cluster basis. That is, the standard selection probabilities for all therapies associated with MTs belonging to the cluster to which the MT associated with the therapy confirmed as effective belongs can be increased by a predetermined amount. Other therapies belonging to the cluster to which the effective therapy belongs are also similarly considered highly effective, and therefore the selection probability of the entire cluster is increased. For example, in a case where, as a treatment for the behavioral MT ID “eatAtOnce”, the effectiveness of treatment by the therapy ID “trush” is confirmed, then the selection probabilities of all therapies associated with behavioral MTs are increased.

Further, a cluster factor for an individual patient can also be modified on the basis of the effectiveness information. For example, the cluster factor of a cluster to which a medical trait associated with a therapy confirmed as effective belongs is increased and, in a case where there is no effect, is decreased. Whether a treatment for any cluster is effective may differ according to the patient. Because the cluster to which the medical trait associated with an effective therapy belongs is likely a cluster effective for that patient, increasing the cluster factor increases the selection probability of a therapy for that cluster, enabling a more effective treatment.

Next, the control unit 501 of the server 130 determines whether the goal behavioral MT has been sufficiently modified (S724) and, in a case where it is determined that the goal behavioral MT has been sufficiently modified, ends the treatment for this goal behavioral MT, returns to the goal behavioral medical trait selection step (S706), and selects a new goal behavioral MT, and the subsequent processing is repeated. For example, in a case where the state of the goal behavioral MT becomes “1: Strongly disagree”, it can be determined that the goal behavioral MT has been sufficiently modified.

In a case where it is determined that the goal behavioral MT has not been sufficiently modified, the control unit 501 returns to the Therapy Selection Probability Table generation step (S708) and updates the Therapy Selection Probability Table, and then the subsequent processing is repeated. Since the Medical Trait State Table of the patient and the selection probabilities have been updated according to the therapies already executed by the patient (S720 to S722), the Therapy Selection Probability Table is updated on the basis of the updated Medical Trait State Table and selection probabilities. The medical trait states for the knowledge-related MT and the cognitive MT sufficiently modified by therapies already executed indicate favorable states, and thus the selection probabilities of therapies for these are changed to lower values, and a therapy for an MT still in an undesirable state is preferentially selected.

With use of the present embodiment, medical traits are clustered into behavioral MTs, knowledge-related MTs, and cognitive MTs and stored in association with therapies suitable for the MTs, and the state of the patient with respect to each trait is stored, making it possible to select a therapy appropriate for the cause of the undesirable behavior of the patient and effectively modify the behavior of the patient to a desired behavior. Furthermore, the effectiveness information of treatments is acquired and the selection probability of the therapy is changed on the basis of this effectiveness information, making it possible to provide a more effective therapy. Furthermore, because the user terminal of the patient is used, an appropriate therapy based on an ever-changing state of the patient can be provided.

### Second Embodiment

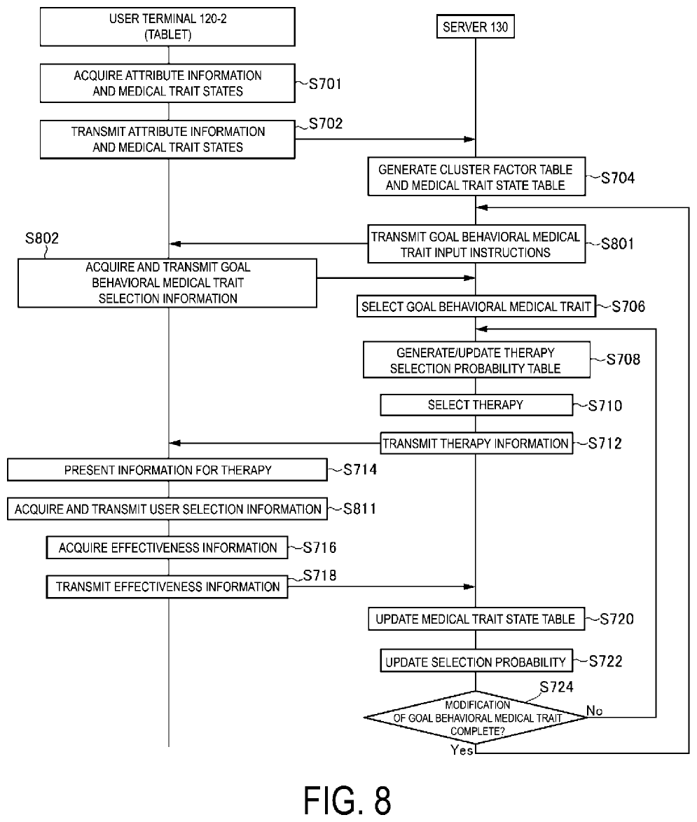

The second embodiment differs from the first embodiment in that the user of the user terminal 120 is the healthcare provider and the embodiment includes S801, S802, and S811 as illustrated in FIG. 8. Hereinafter, the differences from the first embodiment will be mainly described.

In the present embodiment, a user terminal 120-2 is a tablet used by the healthcare provider, but may be another electronic device such as a smartphone or a computer. The healthcare provider inputs, via the user terminal 120-2 of the healthcare provider, the attributes and medical trait states of the patient acquired during interaction with the patient (S701), and transmits the information to the server 130 (S702).

The server 130 generates a Cluster Factor Table and a Medical Trait State Table (S704), and subsequently transmits goal behavioral medical trait input instructions to the user terminal 120-2 (S801). A message prompting input of a goal behavioral MT for the patient is displayed on the display unit 402 of the user terminal 120-2 that receives the instructions, a goal behavioral MT is then selected by the healthcare provider, and the goal behavioral medical trait selection information is transmitted to the server 130 (S802). The server 130, selects a goal behavioral medical trait on the basis of the information (S706), and generates a Therapy Selection Probability Table on the basis of the selected goal behavioral MT (S708).

The control unit 501 of the server 130 selects a therapy on the basis of the Therapy Selection Probability Table in the same way as in the first embodiment (S710), and transmits the therapy information (S712). The user terminal 120-2 that receives the therapy information presents the information for the therapy on the display unit 402 (S714). In a case where a plurality of therapies are selected by the server 130 and the plurality of therapies are presented on the display unit 402, the healthcare provider selects the therapy to be actually applied to the patient. The user terminal 120-2 acquires user selection information indicating the selected therapy and transmits the user selection information to the server 130 (S811). The healthcare provider then provides, to the patient, guidance based on the therapy selected to be applied to the patient.

After a predetermined period elapses, another medical examination is carried out with the patient to inquire about the effectiveness of the applied treatment, and the effectiveness information is input to the user terminal 120-2 (S716) and transmitted to the server 130 (S718). The server 130 updates the Medical Trait State Table and the selection probabilities on the basis of the user selection information and the effectiveness information (S720, S722). The therapy selected by the healthcare provider on the basis of the user selection information is presumed to be an appropriate therapy, and therefore the standard selection probability of the therapy is increased. That is, the standard selection probability is varied by using the selection by the healthcare provider as instructor information. The standard selection probabilities associated with all therapy IDs of the therapy may be changed, or only the relationship with the MT to be treated may be changed. The standard selection probabilities of all therapies associated with the MTs of the cluster of the MT associated with the user-selected therapy can also be increased.

Then, the control unit 501 of the server 130 determines whether modification of the goal behavioral MT is completed (S724) and, in a case where it is determined that modification is completed, returns to the input instruction transmission step (S801) for determining the next goal behavioral medical trait and, in a case where modification is not completed, returns to the Therapy Selection Probability Table generation step (S708) and updates the Therapy Selection Probability Table, and then the subsequent processing is repeated.

In the present embodiment, because the selection probability of the therapy is changed with selection of the therapy by the healthcare provider serving as instructor information, the selection probability of the therapy considered to be highly effective is increased, making it possible to carry out more effective treatment.

### Third Embodiment

The third embodiment differs from the first and second embodiments in that the disorder to be treated is hypertension. Hereinafter, the differences from the first embodiment and the second embodiment will be mainly described.

The information processing flow in the present embodiment is similar to that of FIGS. 7 and 8, but because the disorder to be treated by behavior change is hypertension, each MT table, the therapy table, and each MT-therapy relationship table are generated in association with hypertension. An example of each table is illustrated below.

TABLE 13
Behavioral MT Table

| Behavioral MT ID | Description | Possible State |
|---|---|---|
| tooMuchSoySource | Tends to add | 5: Strongly agree |
| | a great amount | 4: Somewhat agree |
| | of soy sauce and | 3: Not sure |
| | other sauces | 2: Somewhat disagree |
| | | 1: Strongly disagree |
| sleepLess5Hours | Sleeps 5 hours | 5: Strongly agree |
| | or less | 4: Somewhat agree |
| | | 3: Not sure |
| | | 2: Somewhat disagree |
| | | 1: Strongly disagree |

TABLE 14
Knowledge-Related MT Table

| Knowledge-Related MT ID | Description | Possible State |
|---|---|---|
| tooMuchSoySourceIntent | Thinks that to | 5: Strongly agree |
| | add a lot of soy | 4: Somewhat agree |
| | sauce or other | 3: Not sure |
| | sauce is not a | 2: Somewhat disagree |
| | bad thing | 1: Strongly disagree |
| sleepLess5HoursIntent | Thinks that there | 5: Strongly agree |
| | is not much | 4: Somewhat agree |
| | difference between | 3: Not sure |
| | 5 hours and 6 hours | 2: Somewhat disagree |
| | of sleep | 1: Strongly disagree |

TABLE 15
Cognitive MT Table

| Cognitive MT ID | Description | Possible State |
|---|---|---|
| noSaltyNoTaste | If something | 5: Strongly agree |
| | is not salty, | 4: Somewhat agree |
| | it has no taste | 3: Not sure |
| | | 2: Somewhat disagree |
| | | 1: Strongly disagree |
| noShortSleepProblem | Skimping on | 5: Strongly agree |
| | sleep is not | 4: Somewhat agree |
| | problematic | 3: Not sure |
| | for the body | 2: Somewhat disagree |
| | | 1: Strongly disagree |
| cantChangeTaste | Taste | 5: Strongly agree |
| | preferences | 4: Somewhat agree |
| | cannot be | 3: Not sure |
| | changed | 2: Somewhat disagree |
| | | 1: Strongly disagree |

TABLE 16
Therapy Table

| Therapy ID | Description |
|---|---|
| saltReducedFood | Try switching to low salt foods |
| sleepMonitoring | Measure how many hours of sleep you are getting |
| decisionFatigue | For sleeping and hypertension, there is a big difference |
| | between 5 hours and 6 hours. |
| doNotExerciseBeforeSleep | Try not exercising before going to bed |
| sodiumEstimate | Try estimating salt content |
| dashi | Try using dashi instead of salt |
| actuallyLack0fSleep | Let's suspect that you actually lack sleep |

TABLE 17
Behavioral MT-Knowledge-Related MT
Relationship Table

| Behavioral MT ID | Knowledge-Related MT ID |
|---|---|
| tooMuchSoySource | tooMuchSoySourceIntent |
| sleepLess5Hours | sleepLess5HoursIntent |

TABLE 18
Behavior-Cognitive MT Relationship Table

| Behavioral MT ID | Cognitive MT ID |
|---|---|
| tooMuchSoySource | noSaltyNoTaste |
| tooMuchSoySource | cantChangeTaste |
| sleepLess5Hours | noShortSleepProblem |

TABLE 19
Behavioral MT-Therapy Relationship Table

| | | Standard |
|---|---|---|
| | | Selection |
| | | Probability |
| Behavioral MT ID | Therapy ID | Factor |
| tooMuchSoySource | saltReducedFood | 1.2 |
| sleepLess5Hours | doNotExerciseBeforeSleep | 1.2 |

TABLE 20
Knowledge-Related MT - Therapy Relationship Table

| | | Standard Selection |
|---|---|---|
| Knowledge-Related MT ID | Therapy ID | Probability Factor |
| tooMuchSoySourceIntent | sodiumEstimate | 1.0 |
| sleepLess5HoursIntent | sleepMonitoring | 0.8 |

TABLE 21
Cognitive MT - Therapy Relationship Table

| | | Standard Selection |
|---|---|---|
| Cognitive MT ID | Therapy ID | Probability Factor |
| noSaltyNoTaste | dashi | 0.8 |
| noShortSleepProblem | actuallyLackOfSleep | 0.9 |
| cantChangeTaste | dashi | 0.9 |

Furthermore, the following table is generated on the basis of these tables as well as the cluster factors and the acquired medical trait states of patient A.

TABLE 22
Medical Trait State Table of Patient A

| | | | | Individual Selection |
|---|---|---|---|---|
| MT ID | MT Type | State | Cluster Factor | Probability Factor |
| tooMuchSoySource | Behavioral MT | 5 | 1.2 | 6.0 |
| sleepLess5Hours | Behavioral MT | 5 | 1.2 | 5.0 |
| tooMuchSoySourceIntent | Knowledge-related MT | 3 | 1.0 | 2.4 |
| sleepLess5HoursIntent | Knowledge-related MT | 4 | 1.0 | 4.0 |
| noSaltyNoTaste | Cognitive MT | 1 | 0.8 | 0.8 |
| noShortSleepProblem | Cognitive MT | 3 | 0.8 | 2.4 |
| cantChangeTaste | Cognitive MT | 1 | 0.8 | 0.8 |

An example of the Therapy Selection Probability Table for patient A for tooMuchSoySauce with tooMuchSoySauce selected as the goal behavioral MT in the present embodiment is illustrated below.

TABLE 23
Therapy Selection Probability Table of Patient A (Behavioral MT = TooMuchSoySauce)

| | | Standard | Individual | Comprehensive | |
|---|---|---|---|---|---|
| | | Selection | Selection | Selection | |
| Knowledge-related | | Probability | Probability | Probability | Selection |
| MT/Cognitive MT ID | Therapy ID | Factor | Factor | Factor | Probability |
| — | saltReducedFood | 1.2 | 6.0 | 7.2 | 0.657 |
| tooMuchSoySourceIntent | sodiumEstimate | 1.0 | 2.4 | 2.4 | 0.219 |
| noSaltyNoTaste | dashi | 0.8 | 0.8 | 0.64 | 0.058 |
| cantChangeTaste | dashi | 0.9 | 0.8 | 0.72 | 0.066 |

In the same way as in the first and second embodiments, a therapy is selected on the basis of the Therapy Selection Probability Table, and the therapy information associated with the treatment is presented on the user information terminal 120.

### Fourth Embodiment

The fourth embodiment differs from the first to third embodiments in that the disorder to be treated is a psychiatric disorder (depression). Hereinafter, the differences from the first to third embodiments will be mainly described.

The information processing flow in the present embodiment is similar to that of FIGS. 7 and 8, but because the disorder to be treated by behavior change is a psychiatric disorder (depression), each MT table, the therapy table, and each MT-therapy relationship table are generated in association with the psychiatric disorder (depression). An example of each table is illustrated below.

TABLE 24
Behavioral MT Table

| Behavioral MT ID | Description | Possible State |
|---|---|---|
| eatNotEnough | Does not eat well | 5: Strongly agree |
| | | 4: Somewhat agree |
| | | 3: Not sure |
| | | 2: Somewhat disagree |
| | | 1: Strongly disagree |
| homePrison | Does not leave the house | 5: Strongly agree |
| | | 4: Somewhat agree |
| | | 3: Not sure |
| | | 2: Somewhat disagree |
| | | 1: Strongly disagree |

TABLE 25
Knowledge-Related MT Table

| Knowledge- | | |
|---|---|---|
| Related MT ID | Description | Possible State |
| homePrisonIntent | Thinks that there is no | 5: Strongly agree |
| | need to leave | 4: Somewhat agree |
| | the house | 3: Not sure |
| | | 2: Somewhat disagree |
| | | 1: Strongly disagree |
| eatNotEnoughIntent | Thinks that there is no | 5: Strongly agree |
| | need to eat well | 4: Somewhat agree |
| | (Sweets will do) | 3: Not sure |
| | | 2: Somewhat disagree |
| | | 1: Strongly disagree |

TABLE 26
Cognitive MT Table

| Cognitive MT ID | Description | Possible State |
|---|---|---|
| cauzIAmDepressed | Gives up, blaming | 5: Strongly agree |
| | depression | 4: Somewhat agree |
| | | 3: Not sure |
| | | 2: Somewhat disagree |
| | | 1: Strongly disagree |
| anyHow | No matter what, | 5: Strongly agree |
| | nothing changes | 4: Somewhat agree |
| | | 3: Not sure |
| | | 2: Somewhat disagree |
| | | 1: Strongly disagree |
| mindReading | People around me think | 5: Strongly agree |
| | poorly of me and that I | 4: Somewhat agree |
| | am no good | 3: Not sure |
| | | 2: Somewhat disagree |
| | | 1: Strongly disagree |
| zeroSum | When I fail at one thing, | 5: Strongly agree |
| | I feel I will fail at all | 4: Somewhat agree |
| | things and give up | 3: Not sure |
| | | 2: Somewhat disagree |
| | | 1: Strongly disagree |
| statusQuo | Never did so before, so it is | 5: Strongly agree |
| | simply fine to | 4: Somewhat agree |
| | continue as is | 3: Not sure |
| | | 2: Somewhat disagree |
| | | 1: Strongly disagree |

TABLE 27
Therapy Table

| Therapy ID | Description |
|---|---|
| makeJoyEating | Create enjoyment by eating good food |
| MakeJoyPlanning | Plan excursions that you will look forward to doing |
| freewillOfGoingIsJustIllusion | You do not go out because you are motivated, you go out to |
| | get motivated |
| freewillOfEatingIsJustIllusion | You do not eat because you want to eat, you eat and cook to |
| | increase your appetite |
| imageToBe | Imagine yourself as who you want to be |
| mindCold | Depression can happen to anyone and everyone can be cured |
| autoThought | Change your thinking: things may seem bad, but nothing bad |
| | has actually happened |
| sunBathing | Soak in the sun |

TABLE 28
Behavioral MT - Knowledge-Related MT Relationship Table

| Behavioral MT ID | Knowledge-Related MT ID |
|---|---|
| homePrison | homePrisonIntent |
| eatNotEnough | eatNotEnoughIntent |

TABLE 29
Behavioral - Cognitive MT Relationship Table

| Behavioral MT ID | Cognitive MT ID |
|---|---|
| homePrison | cauzIAmDepressed |
| homePrison | anyHow |
| homePrison | mindReading |
| homePrison | zeroSum |
| homePrison | statusQuo |
| eatNotEnough | cauzIAmDepressed |
| eatNotEnough | statusQuo |

TABLE 30
Behavioral MT - Therapy Relationship Table

| | | Standard Selection |
|---|---|---|
| Behavioral MT ID | Therapy ID | Probability Factor |
| eatNotEnough | makeJoyEating | 1.2 |
| homePrison | MakeJoyPlanning | 1.2 |
| homePrison | sunBathing | 1.2 |

TABLE 31
Knowledge-Related MT - Therapy Relationship Table

| Knowledge- | | Standard Selection |
|---|---|---|
| Related MT ID | Therapy ID | Probability Factor |
| homePrisonIntent | freewillOfGoingIsJustIllusion | 1.0 |
| eatNotEnoughIntent | freewillOfEatingIsJustIllusion | 8.0 |

TABLE 32
Cognitive MT - Therapy Relationship Table

| | | Standard Selection |
|---|---|---|
| Cognitive MT ID | Therapy ID | Probability Factor |
| cauzIAmDepressed | mindCold | 0.9 |
| anyHow | smallStepCommitment | 0.9 |
| mindReading | autoThought | 0.7 |
| zeroSum | comfirmEvidence | 0.7 |
| statusQuo | imageToBe | 0.6 |

Furthermore, the following table is generated on the basis of these tables as well as the cluster factors and the acquired medical trait states of patient A.

TABLE 33
Medical Trait State Table of Patient A

| | | | Cluster | Individual Selection |
|---|---|---|---|---|
| MT ID | Cluster Type | State | Factor | Probability Factor |
| eatNotEnough | Behavioral MT | 5 | 1.2 | 6.0 |
| homePrison | Behavioral MT | 5 | 1.2 | 6.0 |
| homePrisonIntent | Knowledge- | 3 | 1.0 | 3.0 |
| | related MT | | | |
| eatNotEnoughIntent | Knowledge- | 4 | 1.0 | 4.0 |
| | related MT | | | |
| cauzIAmDepressed | Cognitive MT | 1 | 0.8 | 0.8 |
| anyHow | Cognitive MT | 3 | 0.8 | 2.4 |
| mindReading | Cognitive MT | 1 | 0.8 | 0.8 |
| zeroSum | Cognitive MT | 1 | 0.8 | 0.8 |
| statusQuo | Cognitive MT | 1 | 0.8 | 0.8 |

An example of the Therapy Selection Probability Table for Patient A for homePrison with homePrison selected as the goal behavioral MT in the present embodiment is illustrated below.

TABLE 34
Therapy Selection Probability Table of Patient A (Behavioral MT = homePrison)

| | | Standard | Individual | Comprehensive | |
|---|---|---|---|---|---|
| Knowledge-related | | Selection | Selection | Selection | |
| MT/Cognitive MT | | Probability | Probability | Probability | Selection |
| ID | Therapy ID | Factor | Factor | Factor | Probability |
| — | makeJoyEating | 1.2 | 6.0 | 7.2 | 0.329 |
| — | sunBathing | 1.2 | 6.0 | 7.2 | 0.329 |
| homePrisonIntent | freewillOfGoing | 1.0 | 3.0 | 3.0 | 0.137 |
| | IsJustIllusion | | | | |
| cauzIAmDepressed | mindCold | 0.9 | 0.8 | 0.72 | 0.033 |
| anyHow | smallStepCommitment | 0.9 | 2.4 | 2.16 | 0.099 |
| mindReading | autoThought | 0.7 | 0.8 | 0.56 | 0.026 |
| zeroSum | comfirmEvidence | 0.7 | 0.8 | 0.56 | 0.026 |
| statusQuo | imageToBe | 0.6 | 0.8 | 0.48 | 0.022 |

In the same way as in the first to third embodiments, a therapy is selected on the basis of the Therapy Selection Probability Table, and the therapy information associated with the treatment is presented on the user information terminal 120.

Similarly, for other disorders treatable by behavior change as well, the present invention can be implemented using a similar information processing flow by preparing a table associated with the disorder.

Further, while the functions of the user terminal 120-1 of the patient, the user terminal 120-2 of the healthcare provider, and the server 130 have been described in the embodiments described above, these functions can be provided and implemented by any of the devices included in the system according to the present invention. For example, in the first embodiment, each table stored in the storage unit 504 of the server 130 can be stored in the storage unit 404 of the user terminal 120-1, and all functions of the server 130 can be performed by the user terminal 120-1.

The embodiments of the present invention have been described for illustrative purposes, but the present invention is not limited to these embodiments. The present invention can be implemented in various forms without departing from the spirit thereof.

### REFERENCE SIGNS LIST

100 System
110 Network
120 User information terminal
120 User terminal
130 Server
201 Processing device
202 Display device
203 Input device
204 Storage device
205 Communication device
206 Program
208 Bus
209 Each program
301 Processing device
302 Display device
303 Input device
304 Storage device
305 Communication device
306 Program
308 Bus
401 Control unit
402 Display unit
403 Input unit
404 Storage unit
405 Communication unit
501 Control unit
502 Display unit
503 Input unit
504 Storage unit
505 Communication unit

1. A system used for treating a disorder treatable by behavior change, the system comprising:
a server; and
a user terminal, wherein
medical traits, associated with a disorder, for indicating medical traits of a patient are clustered into behavioral medical traits, knowledge-related medical traits, and cognitive medical traits,
the server is configured to
store each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait,
select a therapy to be executed from among
the one or more therapies associated with, from among the plurality of behavioral medical traits, a behavioral medical trait selected to be treated, and
therapies associated with knowledge-related medical trait information and cognitive medical trait information associated with the behavioral medical trait thus selected, and
transmit therapy information for the therapy thus selected, and
the user terminal is configured to present information for the therapy on the basis of the therapy information received.

2. The system according to claim 1, wherein
the server is further configured to store a medical trait state indicating a state of each of the medical traits of each patient, a standard selection probability factor for each of the one or more therapies associated with each of the medical traits, and an individual selection probability factor for each of the medical traits of each patient,
the individual selection probability factor for each of the medical traits is determined on the basis of the medical trait state of each of the medical traits of the patient, and a selection probability of the therapy is determined on the basis of the standard selection probability factor for the therapy and the individual selection probability factor for a medical trait of the medical traits associated with the therapy.

3. The system according to claim 2, wherein
the individual selection probability factor is further determined on the basis of a cluster factor, and
the cluster factor is determined per patient for each cluster of the medical traits.

4. The system according to claim 3, wherein
the server is further configured to store attributes of each patient,
the attributes include at least one of a gender, an age, or an occupation, and
the cluster factor is determined on the basis of the attributes.

5. The system according to claim 2, wherein the server is further configured to
acquire effectiveness information indicating whether the medical trait associated with the therapy thus selected has improved,
update the medical trait state of the patient on the basis of the effectiveness information, and
change the individual selection probability factor on the basis of the medical trait state thus updated.

6. The system according to claim 2, wherein the server is further configured to
acquire effectiveness information indicating whether the medical trait associated with the therapy thus selected has improved, and
change a cluster factor of a cluster to which the medical trait belongs on the basis of the effectiveness information.

7. The system according to claim 2, wherein the server is further configured to
acquire effectiveness information indicating whether the medical trait associated with the therapy thus selected has improved, and
change the standard selection probability factor for the therapy thus selected on the basis of the effectiveness information.

8. The system according to claim 2, wherein
the server is configured to, in the selection of the therapy, select two or more of the therapies, and transmit the therapy information for the two or more therapies thus selected,
the user terminal is configured to present information for the two or more therapies on the basis of the therapy information received, and transmit, to a server, user selection information indicating a therapy selected by a user from the two or more therapies of the information presented, and
the server is configured to change at least the standard selection probability factor on the basis of the user selection information.

9. The system according to claim 7, wherein the standard selection probability factor thus changed is the standard selection probability factor for each of the therapies associated with the medical traits belonging to the cluster of the medical traits associated with the therapy thus selected.

10. A server used for treating a disorder treatable by behavior change where medical traits, associated with a disorder, for indicating medical traits of a patient are clustered into behavioral medical traits, knowledge-related medical traits, and cognitive medical traits, the server being configured to
store each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait,
select a therapy to be executed from among
the one or more therapies associated with, from among the plurality of behavioral medical traits, a behavioral medical trait selected to be treated, and
therapies associated with knowledge-related medical trait information and cognitive medical trait information associated with the behavioral medical trait thus selected, and
transmit therapy information for the therapy thus selected.

11. A method executed by a system used for treating a disorder treatable by behavior change where medical traits, associated with a disorder, for indicating medical traits of a patient are clustered into behavioral medical traits, knowledge-related medical traits, and cognitive medical traits, the system including a server and a user terminal, and the server being configured to store each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait, the method comprising the steps of:
selecting, by the server, a therapy to be executed from among
the one or more therapies associated with, from among the plurality of behavioral medical traits, a behavioral medical trait selected to be treated, and
therapies associated with knowledge-related medical trait information and cognitive medical trait information associated with the behavioral medical trait thus selected;
transmitting, by the server, therapy information for the therapy thus selected; and
presenting, by the user terminal, information for the therapy on the basis of the therapy information received to execute the therapy.

12. A method executed by a server used for treating a disorder treatable by behavior change where medical traits, associated with a disorder, for indicating medical traits of a patient are clustered into behavioral medical traits, knowledge-related medical traits, and cognitive medical traits, and the server being configured to store each of a plurality of the behavioral medical traits, a plurality of the knowledge-related medical traits, and a plurality of the cognitive medical traits in association with one or more therapies, the plurality of behavioral medical traits each being further associated with, from among the plurality of knowledge-related medical traits and the plurality of cognitive medical traits, at least a cognitive medical trait, the method comprising the steps of:
selecting, by the server, a therapy to be executed from among
the one or more therapies associated with, from among the plurality of behavioral medical traits, a behavioral medical trait selected to be treated, and
therapies associated with knowledge-related medical trait information and cognitive medical trait information associated with the behavioral medical trait thus selected; and
transmitting, by the server, therapy information for the therapy thus selected.

13. A non-transitory computer-readable computer medium storing a set of programs for causing one or more processor to perform the method recited in claim 11.

14. A non-transitory computer-readable computer medium storing a program for causing a server to perform the method recited in claim 12.
